(19) United States
(12) Patent Application Publication
(10) Pub. No.: US 2011/0004120 A1
(43) Pub. Date: Jan. 6, 2011

(54) BIOPSY DEVICE NEEDLE SET

(75) Inventor: Lev Drubetsky, Coquitlam (CA)

Correspondence Address: IPHORGAN, LTD., 1130 LAKE COOK ROAD, SUITE 240, BUFFALO GROVE, IL, 60089, US

(73) Assignee: ANGIOTECH PHARMACEUTICALS INC., Vancouver (CA)

(21) Appl. No.: 12/790,609

(22) Filed: May 28, 2010

Related U.S. Application Data
(60) Provisional application No. 61/182,695, filed on May 30, 2009.
(60) Provisional application No. 61/181,933, filed on May 28, 2009.

(51) Int. Cl. A61B 10/02

(52) U.S. Cl. 600/567

(57) ABSTRACT

A needle set for use in a biopsy device, where the needle set includes a plurality of annularly nested cannulae. Each of the cannulae have a distal end, a proximal end, a lumen, and a cannula body. At least two of the cannulae are joined at their proximal ends. The distal ends of the two joined cannulae are mountable in a biopsy device. At least one of the two joined cannulae further have at least two flexible linkages joining the cannula body to its proximal end. Upon rotating the cannula body around its longitudinal axis while fixing the proximal end, the linkages present therebetween will contact one another, effectively cutting the luminal space between the cannula body and the proximal end of the cannula, thereby also cutting any material, for example biopsy material, present in the lumen.

### CROSS REFERENCE TO RELATED APPLICATIONS

This application claims the benefit under 35 U.S.C. §119(e) of U.S. Provisional Patent Application Nos. 61/181,933, filed May 28, 2009, and 61/182,695, filed May 30, 2009, which applications are incorporated herein by reference in their entirety.

### FIELD OF INVENTION

The present invention relates generally to needle sets for biopsy devices.

### BACKGROUND OF THE INVENTION

It is often desirable and frequently absolutely necessary to sample or test a portion of tissue from humans and even animals to aid in the diagnosis and treatment of patients with cancerous tumors, pre-malignant conditions and other diseases or disorders. Typically in the case of cancer or the suspicion of malignant tumors, a very important process called tissue biopsy is performed to establish whether cells are cancerous.

Biopsy may be done by an open or closed technique. Open biopsy removes the entire tissue mass or a part of the tissue mass. Closed biopsy on the other hand is usually performed with a needle-like instrument and may be either an aspiration (hollow needle on a syringe) or a core biopsy (special tissue cutting needle design). In needle aspiration biopsy, individual cells or clusters of cells are obtained for cytologic examination. In core biopsy, a segment of tissue is obtained for histologic examination which may be done as a frozen section or paraffin section.

The methods and procedures of obtaining tissue samples for cytologic or histologic examination have been performed historically by manual insertion and manipulation of the needle. These procedures are performed “blind” by the physician and guided by “feel” and known anatomic “landmarks”.

Tumors are first noted in a patient by one of three ways, palpation, x-ray imaging or ultrasound imaging. Once a tumor is identified, a biopsy procedure is performed. Modern medical opinion dictates early detection of cancer, which increases the likelihood of successful treatment. Biopsy are performed on “Tumor Masses” as small as 2 millimeters in diameter. This procedure is performed under ultrasound or x-ray guidance. Tumors of this size cannot be biopsied reliably by hand since the tumor is about the same size as the biopsy needle. Manual attempts at biopsy can push the tumor away without piercing the mass. Automatic puncture devices are needed to accelerate the needle at such a velocity that even a small tumor can be pierced.

Two very important innovations in the field of medical technology have influenced the field of tissue biopsy in the last five years. One, the use of tissue imaging devices which allow the physician to “see” inside the body and visually guide the needle to the tumor mass. Two, the invention of the Automatic Core Biopsy Device (ACBD) or “Biopsy Gun”. The ACBD is an instrument which propels a needle set with considerable force and speed to pierce the tumor mass and collect the tissue sample. This ACBD device has allowed physicians to test tissue masses in the early stages of growth and has contributed to the medical trend of early diagnosis and successful treatment of cancer.

Examples of such ACBD devices have been described with respect to the collection of tissue samples in U.S. Pat. Nos. 4,651,752, 4,702,260, and 4,243,048.

There are several issues with conventional biopsy devices. The most serious one is a failure to obtain a sample during the biopsy device operation cycle. Such sampling unreliability can result from several different factors, including an insufficiently sharp cutting edge, excessive friction in the sampling cannula, and/or a bad sample cutting/retaining mechanism. For example, it has been reported that if a biopsy device operator rotates the device right after firing it, sampling reliability increases dramatically. Such reports suggest that bad sample cutting/retaining mechanisms may contribute to sampling failure.

Additionally, existing biopsy devices may cut only part of a tissue sample's cross-sectional area, which can require in tearing of the remaining part of the sample's cross-sectional area, which may then result in potential loss of part of the sample.

Moreover, conventional biopsy devices tend to have sensitive and easily damaged components that are exposed to strong forces associated with tissue puncture and placement of the needle set into the sample retrieval site as well as with retrieving the biopsy samples. As such, damage can occur to conventional biopsy devices rendering them less effective or ineffective. For example, some existing biopsy devices include windows or other complex structures in the needle set that can trap tissue in them, making biopsy sample capture and extraction difficult.

Thus, it is an objective of the current invention to provide a biopsy device with a more reliable cutting mechanism.

### BRIEF SUMMARY OF THE INVENTION

In one aspect, there is provided a needle set for use in a biopsy device, the needle set including a plurality of annularly nested cannulae, each of said cannulae having a distal end, a proximal end, a lumen, and a cannula body, at least two of said cannulae being joined at their proximal ends, the distal ends of the at least two joined cannulae being mountable in the biopsy device, and at least one of the at least two joined cannulae further comprising at least two flexible linkages joining the its cannula body to it proximal end.

In certain embodiments, at least one cannula in the plurality of cannulae includes a cutting edge at its proximal end.

In certain embodiments, the needle set further includes a stylet annularly nested in the plurality of cannulae.

In certain embodiments, the cannula that includes flexible linkages is disposed in the lumen of the cannula to which it is joined.

In certain embodiments, the needle set is further provided with a therapeutic composition.

In another aspect, there is provided a method of obtaining a biopsy sample.

These and other aspects of the present invention will become evident upon reference to the following detailed description and attached drawings. In addition, various references are set forth herein which describe in more detail certain procedures and/or compositions (e.g. devices and their components), and these references are incorporated herein by reference in their entirety.

### DESCRIPTION OF THE DRAWINGS

FIG. 1 is an exploded perspective view of the proximal ends of inner and outer cannulae of a biopsy needle set in accordance with an embodiment of the invention.

FIG. 2 is a an exploded perspective view of the proximal ends of inner and outer cannulae of a biopsy needle set in accordance with another embodiment of the invention.

FIG. 3 is a cross-sectional perspective view of a needle set mounted in a biopsy device in accordance with an embodiment of the invention.

FIG. 3A is an enlarged cross-sectional perspective view of the proximal ends of the inner and outer cannulae of a biopsy needle.

FIG. 4 is a cross-sectional perspective view of a needle set mounted in a biopsy device when the device is in a cocked position in accordance with an embodiment of the invention shown in FIG. 3.

FIG. 5 is a cross-sectional perspective view from above of a needle set mounted in a biopsy device when the device is in a fired position in accordance with an embodiment of the invention shown in FIG. 3.

FIG. 6 is a cross-sectional perspective view from below of a needle set mounted in a biopsy device when the device is in a fired position in accordance with an embodiment of the invention shown in FIG. 3.

FIG. 7 is a cross-sectional view from below of a needle set mounted in a biopsy device after a first cocking stroke in accordance with an embodiment of the invention shown in FIG. 3.

FIG. 8 is a cross-sectional view from below of a needle set mounted in a biopsy device after a second cocking stroke in accordance with an embodiment of the invention shown in FIG. 3.

FIG. 9 is an exploded view of a biopsy device including a needle set in accordance with an embodiment of the invention shown in FIG. 3.

FIG. 10 is a perspective view of a needle set mounted in a biopsy device in accordance with another embodiment of the invention.

FIG. 11 is a cross-sectional perspective view of a biopsy device in accordance with an embodiment of the invention shown in FIG. 10.

FIG. 12 is a cross-sectional view from below of a needle set mounted in a biopsy device after a second cocking stroke in accordance with an embodiment of the invention shown in FIG. 10.

FIG. 13 is an exploded view of a biopsy device in accordance with an embodiment of the invention shown in FIG. 10.

FIG. 14 is an exploded view of the inner cannula driver and the outer cannula driver.

FIG. 15 is a perspective view of the inner cannula driver and the outer cannula driver in a coupled arrangement.

### DESCRIPTION OF THE INVENTION

Indications of lateral direction such as ‘from above’, ‘upper’, ‘lower’ are defined by the views of the Figures. The terms “proximal” and “distal” refer to the person from whom a biopsy sample is extracted; the terms “front” and “rear” have a corresponding meaning. Thus, the proximal end of a biopsy apparatus is its front end, pointing to the patient.

Embodiments of the needle set disclosed herein include an inner cannula and an outer cannula, the cannulae being arranged substantially coaxially and being joined at or near their respective tips. In various embodiments, the needle set may also include a stylet, while in various other embodiments a stylet is provided in a biopsy device handle into which such needle set embodiments are loaded; in either case, inner and outer cannulae are annularly arranged around a stylet.

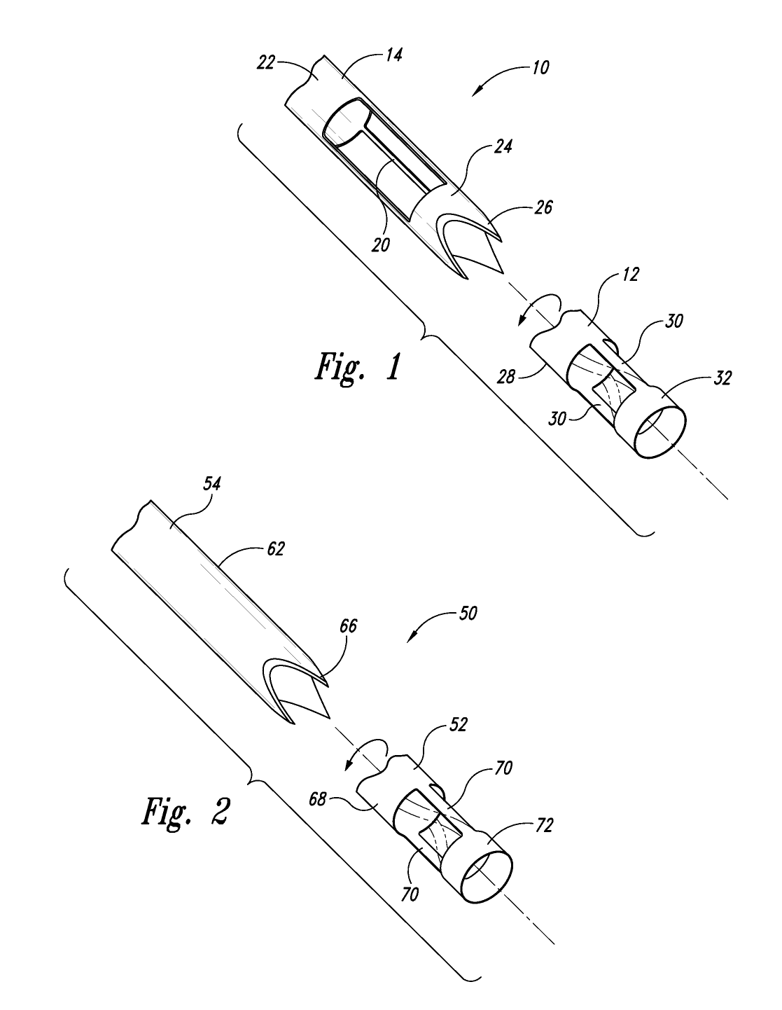

Referring to FIG. 1, inner cannula 12 of needle set 10 has at least two longitudinal webs or linkages 30 linking inner cannula body 28 to inner cannula proximal end 32. Similarly, outer cannula 14 of needle set 10 includes at least two webs or linkages 20 linking outer cannula body 22 to its proximal end 24, at the edge of which is outer cannula tissue cutting edge 26. At least a portion of inner cannula 12 is disposed inside outer cannula 14, and proximal end 32 of inner cannula 12 is joined to proximal end 24 of outer cannula 14. Outer cannula linkages are selected to be sufficiently rigid to prevent substantial rotation of outer cannula proximal end 24 relative to outer cannula body 22. On the other hand, linkages 30 of inner cannula 12 are selected to permit rotation (without damaging linkages 30) of proximal end 32 relative to inner cannula body 28 upon application of a rotational force of a given magnitude, which magnitude would be that provided by the firing of a biopsy device in which the needle set is mounted; such selection may take into account factors such as material choice (for example, in some embodiments the linkages may be made of a sufficiently flexible material to permit such rotation), dimension choice (for example, in some embodiments the linkages may be sufficiently narrow to permit such rotation), and geometry choice (for example, in some embodiments the linkages may include ball joints, flexural joints, etc., joining them to the cannula body and cannula end). Thus, in some embodiments, when a distal portion of inner cannula body 28 is held rotationally immobile while outer cannula 14 is rotated about its axis, then inner cannula proximal end 32 rotates with outer cannula 14, causing inner cannula linkages 30 to twist and approach (and, if desired, meet or pass) each other at a point of intersection. Alternatively, in other embodiments, when outer cannula 14 is held rotationally immobile while a distal portion of inner cannula body 28 is rotated about its axis, then inner cannula proximal end 32 remains immobile with outer cannula 14, resulting in a rotation of inner cannula proximal end 32 relative to inner cannula body 22 and causing inner cannula linkages 30 to twist and approach (and, if desired, meet or pass) each other at a point of intersection. The minimum degree of rotation required to cause inner cannula linkages 30 to approach each other at an intersection point will depend on the width of linkages provided; for example, in embodiments having very thin inner cannula linkages, about 180° of rotation is required to cause the linkages to meet at an intersection point, whereas in embodiments in which wider linkages are provided, less rotation may be required to twist the linkages into meeting.

The material choice, geometry and/or dimensions of inner cannula linkages 30 are also selected to allow linkages 30 to sever tissue. For example, in some embodiments linkages 30 may include a sharp longitudinal edge to sever tissue. Similarly, in some embodiments, linkages 30 may be thin enough to sever tissue. In yet other embodiments, linkages 30 may be provided with (e.g., by being coated with or otherwise incorporating) a cauterising agent, or be heated or provided with an electric current to cauterisingly sever tissue. When needle set 10 is loaded into a biopsy device and deployed to a tissue site, cutting edge 26 of outer cannula 14 extends into and cuts through the tissue, capturing a volume of tissue circumscribed by the cutting edge 26 into the lumen of inner cannula 12. As needle set 10 reaches its full extension into the tissue site, outer cannula 14 is rotated about its axis, causing proximal end 32 of inner cannula 12 to rotate relative to inner cannula body 22, which in turn twists inner cannula linkages 30 until they meet, pinching and/or severing tissue through which linkages 30 pass along the way. The volume of tissue inside the lumen of inner cannula 12 and cut by the twisting of linkages 30 defines the biopsy sample. Tissue that is excluded from the biopsy sample may escape from the needle set either through the open proximal end 24 of outer cannula 14 or through the apertures defined by outer cannula linkages 20. When needle set 10 is withdrawn with its biopsy sample from the tissue site, the biopsy sample may be released by reversing the rotation of outer cannula 14 until inner cannula linkages return to their original position.

Referring now to FIG. 2, needle set 50 includes inner cannula 52 and outer cannula 54. Inner cannula 52 includes at least two longitudinal webs or linkages 70 linking inner cannula body 68 to inner cannula proximal end 72, while outer cannula 54 includes outer cannula body 62 and outer cannula tissue cutting edge 66 at its proximal end. At least a portion of inner cannula 52 is disposed inside outer cannula 54, and proximal end 72 of inner cannula 52 is joined to outer cannula 54 near tissue cutting edge 66. Linkages 70 are selected to permit rotation (without damaging linkages 70) of proximal end 72 relative to inner cannula body 68 upon application of a rotational force of a given magnitude, which magnitude would be that provided by the firing of a biopsy device in which the needle set is mounted; again, such selection may take into account factors such as material choice (for example, in some embodiments the linkages may be made of a sufficiently flexible material to permit such rotation), dimension choice (for example, in some embodiments the linkages may be sufficiently narrow to permit such rotation), and geometry choice (for example, in some embodiments the linkages may include ball joints, flexural joints, etc., joining them to the cannula body and cannula end). Thus, in some embodiments, when a distal portion of inner cannula body 68 is held rotationally immobile while outer cannula 54 is rotated about its axis, then inner cannula proximal end 72 rotates with outer cannula 54, causing inner cannula linkages 70 to twist and approach (and, if desired, meet or pass) each other at a point of intersection. Alternatively, in other embodiments, when outer cannula 54 is held rotationally immobile while a distal portion of inner cannula body 68 is rotated about its axis, then inner cannula proximal end 72 remains immobile with outer cannula 54, resulting in a rotation of inner cannula proximal end 72 relative to inner cannula body 62 and causing inner cannula linkages 70 to twist and approach (and, if desired, meet or pass) each other at a point of intersection. The minimum degree of rotation required to cause inner cannula linkages 70 to approach each other at an intersection point will depend on the width of linkages provided; for example, in embodiments having very thin inner cannula linkages, about 180° of rotation is required to cause the linkages to meet at an intersection point, whereas in embodiments in which wider linkages are provided, less rotation may be required to twist the linkages into meeting.

The material choice, geometry and/or dimensions of inner cannula linkages 70 are also selected to allow linkages 70 to sever tissue. For example, in some embodiments linkages 70 may be include a sharp longitudinal edge to sever tissue. Similarly, in some embodiments, linkages 70 may be thin enough to sever tissue. In yet other embodiments, linkages 70 may be provided with (e.g., by being coated with or otherwise incorporating) a cauterising agent, or be heated or provided with an electric current to cauterisingly sever tissue. When needle set 50 is loaded into a biopsy device and deployed to a tissue site, cutting edge 66 of outer cannula 54 extends into and cuts through the tissue, capturing a volume of tissue circumscribed by the cutting edge 66 in the lumen of inner cannula 52. As needle set 50 reaches its full extension into the tissue site, outer cannula 54 is rotated about its axis, causing proximal end 72 of inner cannula 52 to rotate relative to inner cannula body 62, which in turn twists inner cannula linkages 70 until they meet, pinching and severing tissue through which linkages 70 pass along the way. The volume of tissue inside the lumen of inner cannula 52 and cut by the twisting of linkages 70 defines the biopsy sample. Tissue that is excluded from the biopsy sample may escape from the needle set through the open proximal end of outer cannula 54. When needle set 50 is withdrawn with its biopsy sample from the tissue site, the biopsy sample may be released by reversing the rotation of outer cannula 54 until inner cannula linkages return to their original position.

Additionally, biopsy needle sets described herein may be provided with compositions to treat conditions in the tissue, promote healing, and prevent undesirable effects such as scar formation, infection, pain, and so forth. This can be accomplished in a variety of manners, including for example: (a) by directly affixing to the needle set a formulation (e.g., by either spraying the needle set with a polymer/drug film, or by dipping the needle set into a polymer/drug solution), (b) by coating the needle set with a substance such as a hydrogel which will in turn absorb the composition, or (c) constructing the needle set itself with a composition. Such compositions may include without limitation anti-proliferative agents, anti-angiogenic agents, anti-infective agents, fibrosis-inducing agents, anti-scarring agents, lubricious agents, echogenic agents, anti-inflammatory agents, cell cycle inhibitors, analgesics, and anti-microtubule agents. For biopsies conducted to establish the presence or absence of cancerous tissue, providing needle sets with anti-cancer compositions may be of particular interest. Compositions may also include a plurality of agents either together or on different portions of the needle set, where the multiple compositions can be selected either for different purposes (such as combinations of analgesics, anti-infective and anti-scarring agents) or for their synergistic effects.

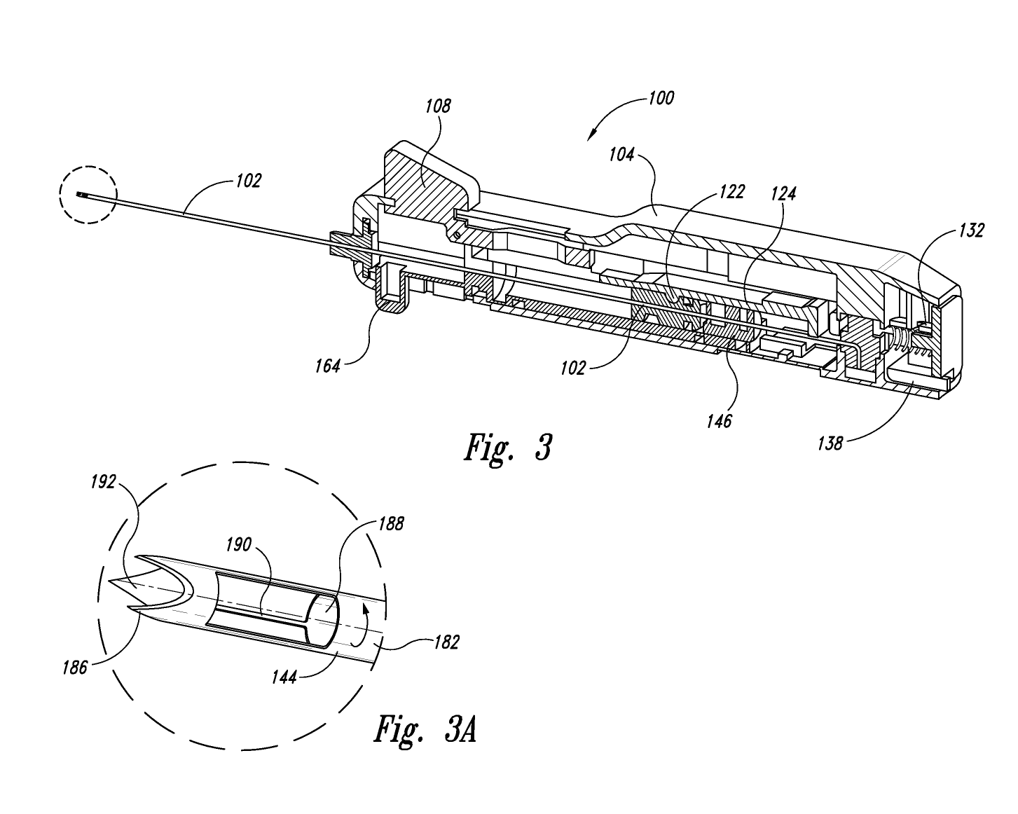

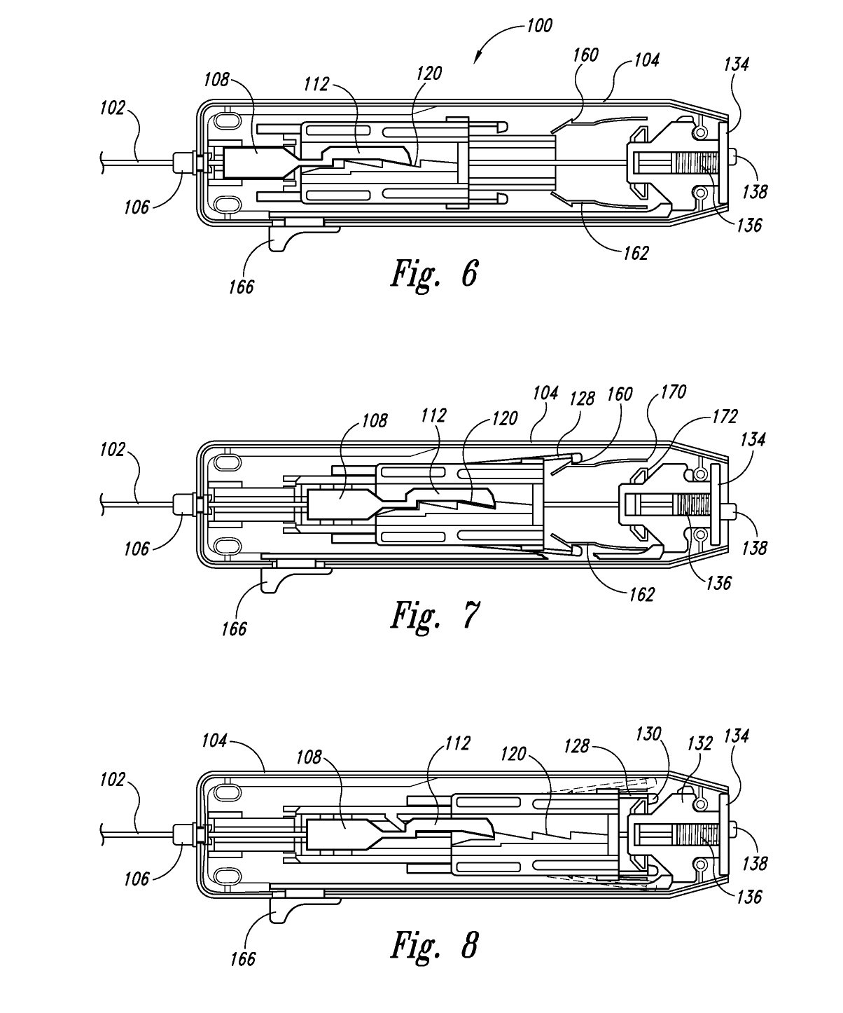

The biopsy device illustrated in FIGS. 3 to 9 includes a biopsy needle set mounted in a biopsy device handle, the needle set having a plurality of nested cannulae extending through the proximal end of the handle. At least two of the cannulae are joined at their proximal ends, and at least one of the so-joined cannulae includes at least two longitudinal webs or linkages joining its proximal end to the rest of its body. The device is provided with a mechanism allowing two cocking strokes to fully cock it, such that the operator of the device may operate it with one hand. In this embodiment, the length of each cocking stroke is selected to correspond to the motion that an operator's digit (on the same hand used the operator to hold the device) may comfortably impart to it, and the sum of the lengths of the cocking strokes is equal to or greater than, depending on the throw depth and whether or not a twisting motion is to be imparted to all or part of the needle set, the distance traveled by the needle set from a fully cocked position to a fully fired position. Where the distance traveled by a biopsy needle set is greater (for example, where throw depths are greater), it may be desirable to provide additional cocking strokes. In yet other embodiments, only one cocking stroke may be required to fully cock the device, whether the operator uses two hands or one. For example, in embodiments for which it is desired to have two-handed operation of a biopsy device, such that one hand of an operator is used to hold the device while the other is used to cock it, a single cocking stroke that is equal or greater in length than the distance traveled by a needle set from full cocking of the device to full firing could be used. In other embodiments, a mechanism allowing for full single-handed cocking of a needle set in a biopsy device may be provided with a gear system or the like.

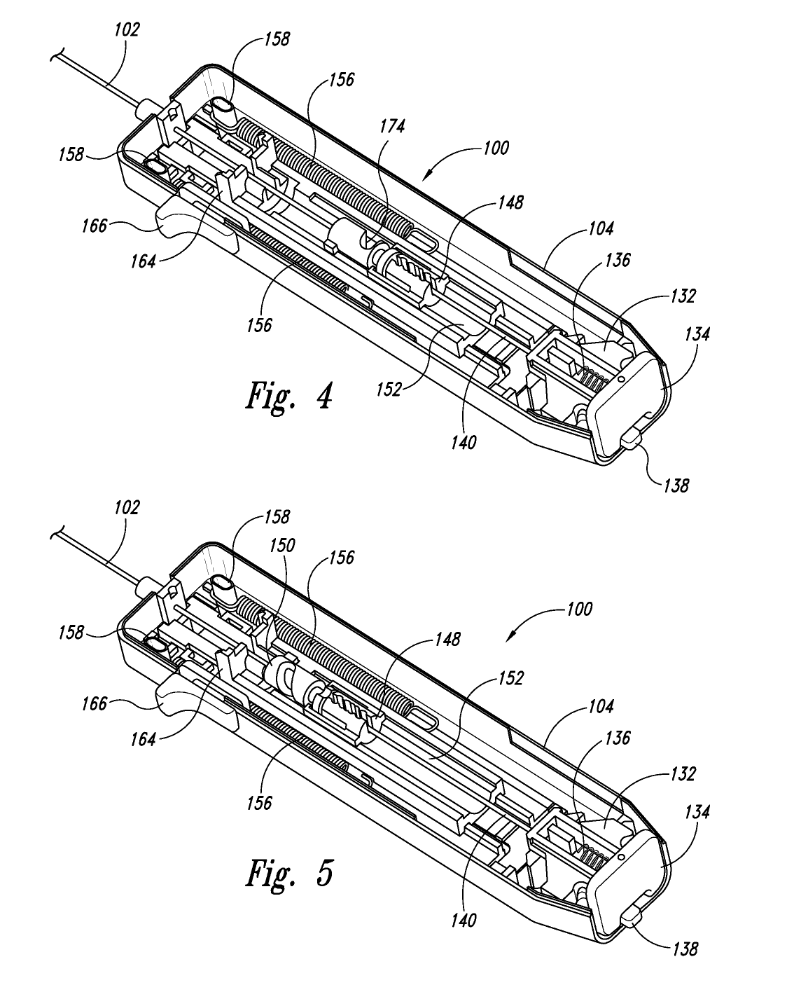

Referring now to FIG. 3, the biopsy device 100 includes a needle set 102, a housing 104, a cocking assembly 108, a carriage assembly 124, an actuator assembly 132, needle set driver assembly 146, and a throw depth stop 164. Referring additionally to FIGS. 4 to 9, cocking actuator 114 of cocking assembly 108 extends out of housing 104 through aperture 118, and may additionally be provided with cocking actuator button 110. Cocking lever 112 of cocking assembly 108 engages one of a plurality of catches 120 of carriage assembly 124. Carriage drive assembly 156, represented in this embodiment as a pair of tension springs affixed at the other of their ends to mounts 158 near the proximal end of the housing 104, engages carriage 126 of assembly 124, which includes carriage arms 128 that, during the first cocking stroke, slidingly engage intermediate catch guides 160 on housing 104. Carriage 126 also engages outer cannula driver 150, to which outer cannula 144 is drivably mounted, and outer cannula driver 150 in turn engages inner cannula driver 148, to which inner cannula 142 is drivably mounted. In certain other embodiments, the carriage may engage another cannula driver, or all such drivers. Inner and outer cannulae of needle set 102 are annularly arranged around a stylet. In the present embodiment, stylet assembly 140 includes a stylet with a stylet mount at one end thereof, disposed distally inside housing 104. (In various other embodiments, the needle set may also include a stylet, thereby obviating the need to provide one in the device handle.) Inner cannula 142 and outer cannula 144 extend through an opening in the proximal end of the housing 104, which may be reinforced with a needle set guide 106 (which needle set guide may be integral part of the housing 104 or mounted thereon) to further stabilize guidance of needle set 102.

In one embodiment, inner cannula driver 148 and outer cannula driver 150 are disposed laterally in series and are coupled by cannula driver coupler 154, thus comprising needle set driver assembly 146; as such, displacement of one cannula driver causes displacement of the other. Both drivers are laterally displaceable along needle set driver guide 152. Needle set driver guide 152 is mounted on an inner surface of housing 104 and includes at its proximal end a barrier, comprising part of throw depth stop 164, which determines the travel distance of outer cannula driver 150 and thereby prevents needle set 102 from traveling past the preselected throw depth.

In the present embodiment, the device is cocked in two strokes, the first of which displaces the carriage from its initial uncocked position laterally through the housing to an intermediate position. Referring to FIG. 7, as the device is being cocked during such a first cocking stroke, cocking assembly 108 is displaced distally as the cocking assembly is actuated by the operator applying force to cocking actuator button 110 covering cocking actuator 114. As cocking assembly 108 moves distally, cocking lever 112 engages one of the distal carriage catches 120 and thereby moves carriage assembly 124 along with it. Resilient carriage arms 128 and carriage arm ends 130 are thus displaced along intermediate catch guides 160 (which, in the present embodiment, are located on the inner surface of housing 104). As intermediate catch guides 160 do not, in the present embodiment, run parallel to a central axis of the housing 104 but rather splay out at their distal ends towards the sides of the handle, carriage arms 128 are deformed slightly to splay out towards the housing 104 as they travel distally along intermediate catch guides 160 during the first cocking stroke. Thus, when carriage arms 128 reach the distal ends of intermediate catch guides 160, carriage arms 128 tend to resile back towards the centre of the handle, to the extent that the continued engagement of carriage arms 128 with intermediate catch guides 160 permit. Once carriage arm ends 130 have moved back towards the center of the handle, the engagement of carriage arm ends 130 with the intermediate catches 162 at the distal end of the intermediate catch guides 160 prevent carriage arms 128 from sliding proximally while carriage drive assembly 156 prevents carriage arms 128 from sliding distally without the application of additional force to displace ends 130 from intermediate catches 162 (as would be provided, for example, with the force of a second cocking stroke). At the same time, referring now to FIG. 4, inner cannula driver 148 and outer cannula driver 150 may travel along needle set driver guide 152 until carriage arm ends 130 reach the intermediate catches 162. This is the intermediate stop, or partially cocked, position.

Once the intermediate stop position is reached and carriage arm ends 130 are engaged in intermediate catches 162, and no more force is applied by the operator to complete the first cocking, cocking repositioner 116 (which, in the present embodiment, is represented by a tension spring attached at one end to the cocking assembly and at its other end to a proximal location on the inner surface of housing 104) urges cocking assembly 108 back to its original, uncocked position.

As, at the end of the first cocking stroke, carriage 126 is displaced distally relative to cocking assembly 108, cocking lever 112 engages one of the proximal cocking catches 120 of carriage assembly 124. As force of the second cocking stroke is applied, cocking lever 112 is again displaced distally and with it displaces carriage 126 further distally, causing the release of carriage arm ends 130 from intermediate catches 162. This time, resilient carriage arms 128 and carriage arm ends 130 are displaced along main catch guides 170 (which, like intermediate catch guides 160, angle away from the central axis of the device housing 104), deforming carriage arms 128 out towards the sides of the housing 104 as they travel distally along main catch guides 170. Carriage arms 128 are thus advanced towards main catches 172, at which carriage arms 128 tend to resile back towards the central axis of the handle, and arm ends 130 are caught in catches 172. Carriage 126 is prevented from moving further distally by tension in the carriage drive assembly 156 and proximally by engagement of arm ends 130 in main catches 172. Displacement of carriage 126 during the second cocking stroke results in the displacement of both outer cannula driver 150 and inner cannula driver 148 further distally along needle set driver guide 152 until carriage arm ends 130 come to rest in main catches 172. This is the fully cocked position; once it is reached and the operator is no longer applying a cocking force, cocking repositioner 116 again returns cocking assembly 108 to its initial position.

As the depth in tissue to which the needle set is to travel may be set using the throw depth selector 166, the position of throw depth selector 166 and throw depth stop 164 limits the distance which inner cannula driver 148 and outer cannula driver 150, and their respective cannulae, may travel proximally. Throw depth stop 164 may be configured to provide any number of throw depths within a given range (which range is determined by the length of the housing opening along which the throw depth selector 166 may be displaced and set), or it may be configured to provide a limited number of throw depths. Throw depth stop 164 is associated with needle set driver guide 152, and displacement of throw depth stop 164 results in displacement of needle set driver guide 152 in housing 104, thus determining how far carriage 126, and therefore inner cannula driver 148 and outer cannula driver 150, may travel within housing 104 to throw depth stop 164 at the proximal end of guide 152. In other embodiments, the throw depth selector may take other configurations, such as a dial assembly.

Once the throw depth selected and the device is fully cocked, the needle set may be injected for tissue sampling at a tissue entry site that the device operator has selected. To actuate the device, the operator moves actuator safety 138 below actuator assembly 132 (which, in the present embodiment, also includes actuator 134 and actuator repositioner 136) from its blocking, or “safe”, position which prevents actuating the device to its actuating position centrally below actuator 134 (as illustrated in FIGS. 4 and 5) and then presses actuator 134, urging carriage arm ends 130 out from main catches 172. After releasing carriage arm ends 130 from main catches 172, actuator 134 is returned to its unactuated position by actuator repositioner 136. The operator's exertion of force against actuator 134 causes it to be displaced proximally towards the carriage arm ends 130 in the main catches 172, and forces the carriage arm ends 130 out of the main catches 172. This release of the carriage arm ends 130 allows the release of potential energy in carriage drive assembly 156 (originally stored by the two-stage cocking of the device), which causes carriage assembly 124, and therefore inner cannula driver 148 and outer cannula driver 150, to travel proximally until outer cannula driver 150 reaches the proximal end of needle set driver guide 152. Accordingly, the proximal ends of inner cannula 142 and outer cannula 144 travel away from the device and towards the tissue sampling site.

To impart a twisting motion to at least one of the cannulae of the needle set, the corresponding cannula driver rotates during actuation of the device. For cylindrical cannula drivers, the driver may, by way of example, be part of a worm drive (wherein the driver includes a worm on its outer surface and the needle set driver guide or carriage assembly includes a worm gear, or vice versa) or, inter alia, be provided with a spiral groove and pin assembly. It is to be understood that various types of rotation-imparting means may be suitably used, the selection and configuration of which may depend on factors such as manufacturing costs, the degree of rotation desired, and the needle set travel distance over which such rotation is to be imparted.

In the embodiment illustrated in FIGS. 3 to 9, inner cannula 142 includes at least two longitudinal webs or linkages 190 linking inner cannula body 188 to inner cannula proximal end 192, while outer cannula 144 includes outer cannula body 182 and outer cannula tissue cutting edge 186 at its proximal end. At least a portion of inner cannula 142 is disposed inside outer cannula 144, and proximal end 192 of inner cannula 142 is joined to outer cannula 144 near tissue cutting edge 186. Linkages 190 are selected to permit rotation (without damaging linkages 190) of inner cannula proximal end 192 relative to inner cannula body 188 upon application of a rotational force by the firing of the biopsy device 100 in which the needle set is mounted. Accordingly, when a distal portion of inner cannula body 188 is held rotationally immobile while outer cannula 144 is rotated about its axis, then inner cannula proximal end 192 rotates with outer cannula 144, causing inner cannula linkages 190 to twist and approach (and, if desired, meet or pass) each other at a point of intersection. The minimum degree of rotation required to cause inner cannula linkages 190 to approach each other at an intersection point will depend on the width of linkages provided; for example, in embodiments having very thin inner cannula linkages, about 180° of rotation is required to cause the linkages to meet at an intersection point, whereas in embodiments in which wider linkages are provided, less rotation may be required to twist the linkages into meeting.

To effect this rotation, outer cannula driver 150 includes a helical groove 174 on its outer surface which slidingly engages pin 122 (shown in FIG. 3) of carriage assembly 124. Thus, when the proximal end of outer cannula driver 144 reaches the distal end of throw depth stop 164 and is prevented from travelling further proximally, carriage assembly 124 continues to move proximally for a distance no greater than the length of helical groove 174 and so causes outer cannula driver 150 to rotate in order to maintain engagement with pin 122. In the course of a biopsy procedure, the rotation of outer cannula driver 150 occurs when the needle set 102 has reached the tissue sampling site and tissue from that sampling site has entered the lumen of the inner cannula 142. Thus, when the rotation of outer cannula driver 150 causes rotation of outer cannula 144 relative to inner cannula body 188, linkages 190 twist and approach one another, severing tissue resulting in a discrete biopsy sample captured in the needle set 102 that can then be retrieved.

In various other embodiments, the helical groove might be provided on a carriage assembly and the pin might be provided on the surface of the cannula driver corresponding to the cannula which is to be twisted. In yet other embodiments, one of the helical groove and pin may be disposed on the cannula driver of interest, while the other of the pair may be disposed on a throw depth stop or needle set driver guide.

Once the device has been actuated and injected into tissue, and a biopsy sample captured by the needle set, the needle set may be withdrawn from the tissue site. To retrieve the biopsy sample from the needle set, the device may be cocked again to draw the needle set back. In the embodiment shown in FIGS. 3 to 9, cocking the device after actuation causes the rotation of outer cannula driver 150 in a direction opposite to that which was caused by actuation, and urges both inner and outer cannula drivers 148 and 150 distally. As inner and outer cannulae 142 and 144 are nestedly arranged over the stylet portion of assembly 140 and as stylet assembly 140 remains stationary, the distal movement of cannula drivers 148 and 150 causes cannulae 142 and 144 to travel distally and the proximal end of stylet assembly 140 to inhibit the corresponding distal movement of the biopsy sample. In this fashion, the distal movement of cannulae 142 and 144 exposes and releases the biopsy sample.

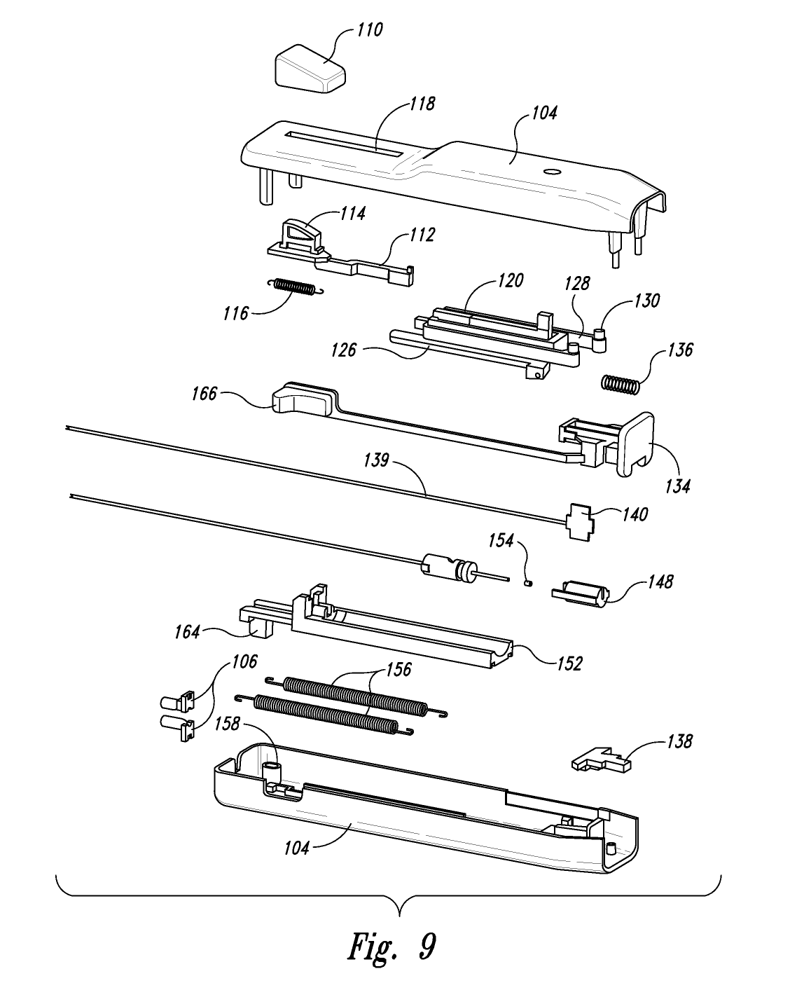

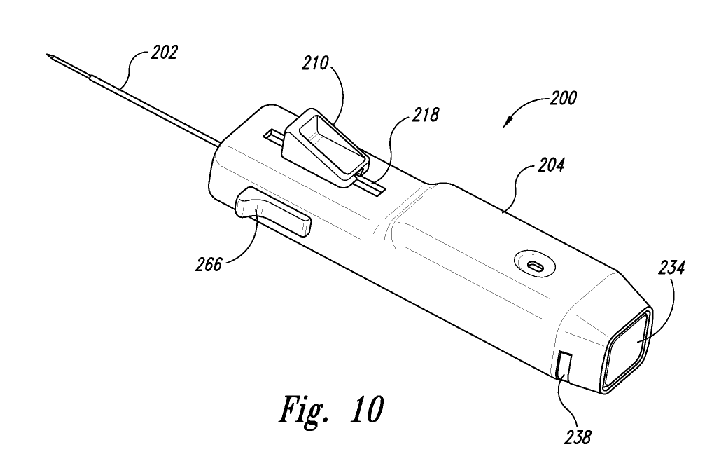

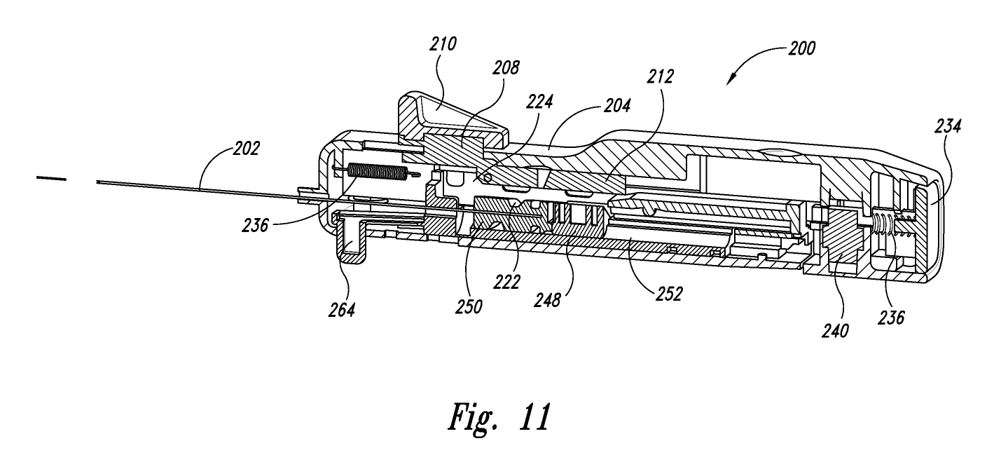

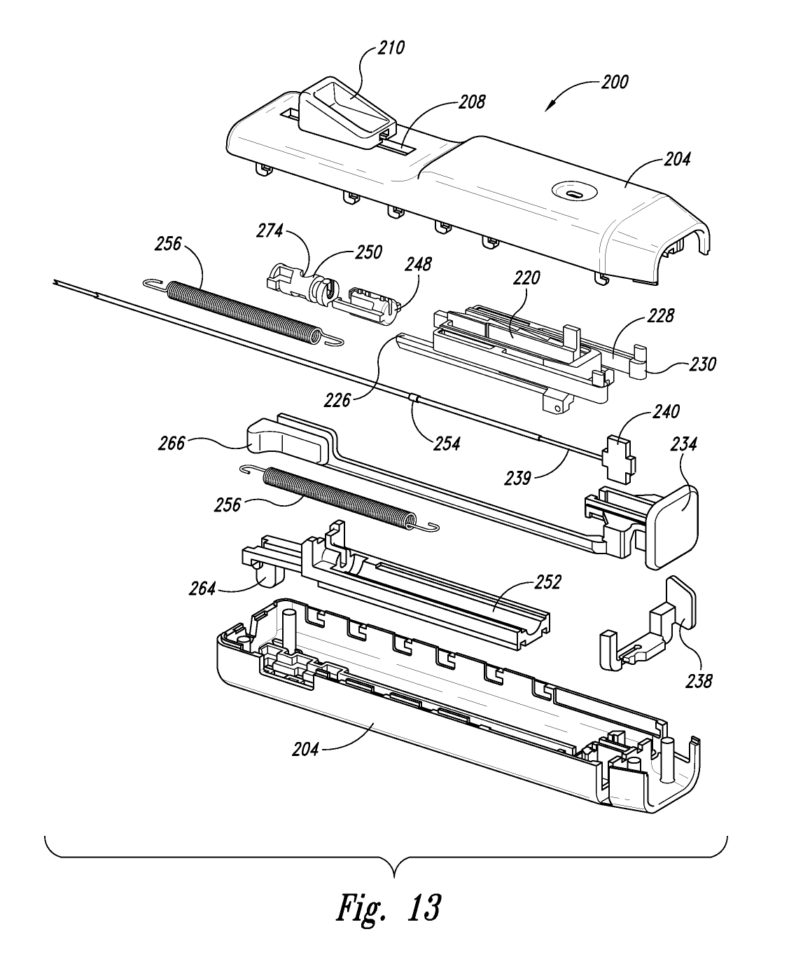

Referring now to the embodiment shown in FIGS. 10 to 13, biopsy device 200 as shown in FIG. 10 includes cocking button 210 slidably disposed in aperture 218 and throw depth selector 266 near the proximal end of housing 204, actuator 234 disposed at the distal end of housing 204, and actuator safety 236 disposed near the distal end of housing 204. Needle set 202 is mounted in device housing 204. Referring additionally to FIGS. 9 to 11, the device is cocked in two strokes, the first of which displaces carriage 226 from its initial uncocked position laterally through housing 204 to an intermediate position. Cocking assembly 208 is displaced distally as it is actuated by the operator applying force to cocking actuator button 210. As cocking assembly 208 moves distally, cocking lever 212 engages one of the distal carriage catches 220 and thereby moves carriage assembly 224 along with it. Resilient carriage arms 228 and carriage arm ends 230 are thus displaced to an intermediate (or partly cocked) position. At the same time, inner cannula driver 248 and outer cannula driver 250, coupled by cannula driver coupler 254, travel along needle set driver guide 252 until carriage arm ends 230 reach the intermediate position. Cocking repositioner 216 (which, in the present embodiment, is represented by a tension spring attached at one end to the cocking assembly and at its other end to a proximal location on the inner surface of housing 204) then urges cocking assembly 208 back to its original, uncocked position.

As, at the end of the first cocking stroke, carriage 226 is displaced distally relative to cocking assembly 208, cocking lever 212 engages one of the proximal cocking catches 220. As force from the second cocking stroke is applied, cocking lever 212 is again displaced distally and with it displaces carriage 226 further distally, along main catch guides 270 until carriage arm ends 230 are caught in main catches 272. Carriage 226 is prevented from moving further distally by tension in the carriage drive assembly 256 and proximally by engagement of arm ends 230 in main catches 272. Displacement of carriage 226 during the second cocking stroke results in the displacement of both outer cannula driver 250 and inner cannula driver 248 further distally along needle set driver guide 252 until carriage arm ends 230 come to rest in main catches 272. This is the fully cocked position; once it is reached and the operator is no longer applying a cocking force, cocking repositioner 216 again returns cocking assembly 208 to its initial position.

As throw depth stop 264 is associated with needle set driver guide 252, and displacement of throw depth stop 264 results in displacement of needle set driver guide 252 in housing 204, the operator may set throw depth stop to the desired depth (that is, the depth of the tissue from which a biopsy sample is desired) in order to determine how far carriage 226, and therefore inner cannula driver 248 and outer cannula driver 250, may travel within housing 204 to throw depth stop 264 at the proximal end of guide 252.

Once the throw depth is selected and the device is fully cocked, the needle set may be injected for tissue sampling at a tissue entry site that the device operator has selected. To actuate the device, the operator moves actuator safety 238 out from its “safe” position preventing proximal displacement of actuator assembly 232 and then presses actuator 234, urging carriage arm ends 230 out from main catches 272. After releasing carriage arm ends 230 from main catches 272, actuator 234 is returned to its unactuated position by actuator repositioner 236. The operator's pressing action causes actuator 234 to be displaced proximally towards the carriage arm ends 230 in the main catches 272, and forces the carriage arm ends 230 out of the main catches 272. This release of the carriage arm ends 230 allows the release of potential energy in carriage drive assembly 256 (originally stored by the two-stage cocking of the device), which causes carriage 226, and therefore inner cannula driver 248 and outer cannula driver 250, to travel proximally until outer cannula driver 250 reaches the proximal end of needle set driver guide 252. Accordingly, the proximal end of needle set 202 travels away from the device and towards the tissue sampling site.

To impart a twisting motion to at least one of the cannulae and thereby facilitate the severing of a tissue sample by needle set 202, the corresponding cannula driver rotates during actuation of the device. For cylindrical cannula drivers, the driver may, by way of example, be part of a worm drive (wherein the driver includes a worm on its outer surface and the needle set driver guide or carriage assembly includes a worm gear, or vice versa) or, inter alia, be provided with a spiral groove and pin assembly. It is to be understood that various types of rotation-imparting means may be suitably used, the selection and configuration of which may depend on factors such as manufacturing costs, the degree of rotation desired, and the needle set travel distance over which such rotation is to be imparted.

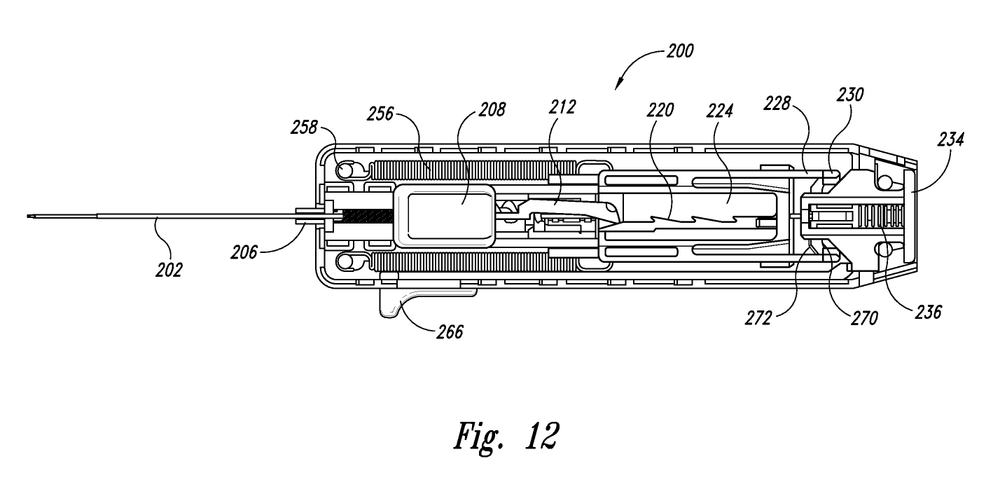

In the embodiment illustrated in FIGS. 10 to 15, outer cannula driver 250 includes a helical groove 274 on its outer surface which slidingly engages pin 222 (shown in FIG. 12) of carriage assembly 224. Thus, when the proximal end of outer cannula driver 244 reaches the distal end of throw depth stop 264 and is prevented from travelling further proximally, carriage assembly 224 continues to move proximally for a distance no greater than the length of helical groove 274 and so causes outer cannula driver 244 to rotate in order to maintain engagement with pin 222. In various other embodiments, the helical groove might be provided on a carriage assembly and the pin might be provided on the surface of the cannula driver corresponding to the cannula which is to be twisted. In yet other embodiments, one of the helical groove and pin may be disposed on the cannula driver of interest, while the other of the pair may be disposed on a throw depth stop or needle set driver guide.

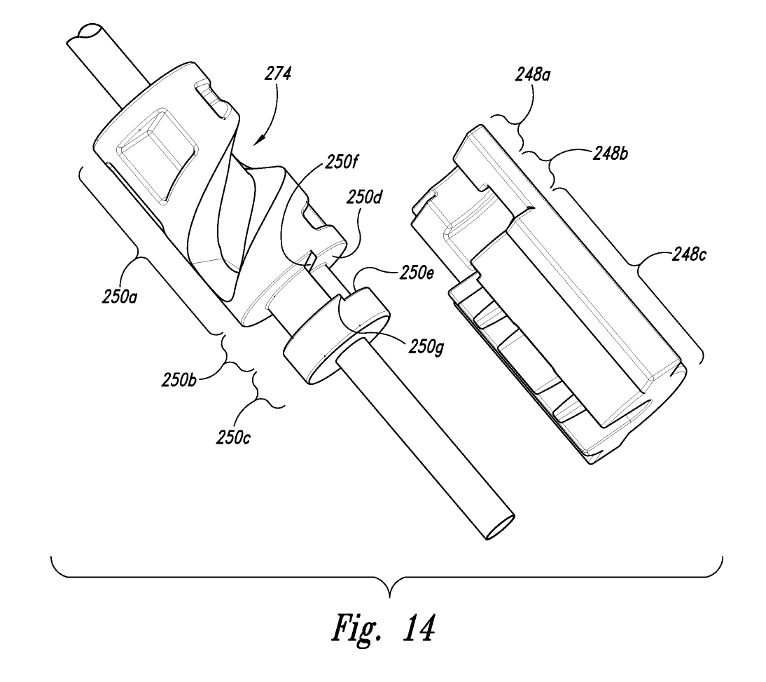

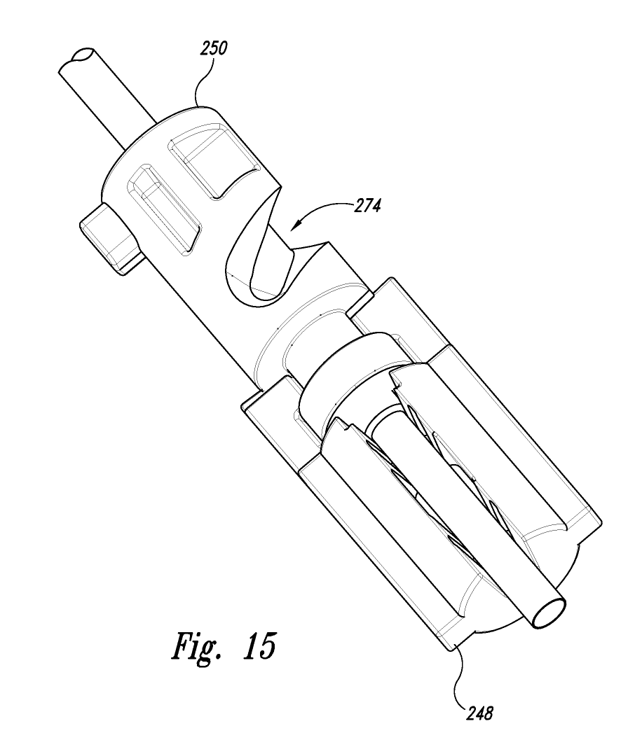

In the embodiment illustrated in FIGS. 14 and 15, the outer cannula driver 250 is coupled to the inner cannula driver 248 via portions of one of the drivers fitting securely into portions of the other driver. Thus, as shown in FIG. 14, outer cannula driver 250 can be divided into regions 250a, 250b, and 250c, while inner cannula driver 248 can be divided into regions 248a, 248b and 248c. In the embodiment shown in FIGS. 14 and 15, region 250b has a smaller diameter than the adjacent regions 250a and 250c, thus providing a neck which fits into region 248a of the inner cannula driver, as best seen in FIG. 15 where the drivers 248 and 250 are shown in a coupled state. In addition, region 248b is configured to accept the relatively large diameter region 250c of the outer cannula driver. The proximal surface 250d and the opposing distal surface 250e of outer cannula 250 each have a pitch, as a screw has a pitch, where the two surfaces 250d and 250e are essentially parallel to one another. Since these two surfaces each have a pitch, there must be a step up (or down, depending on one's perspective) and these steps are shown as features 250f and 250g in FIG. 14. When the region 248a of the inner cannula driver is fitted around the neck 250b of the outer cannula driver, to provide a coupled arrangement, the two drivers can rotate with respect to one another and also move slightly in a longitudinal direction with respect to one another. This movement is desirable in order to compensate for the motion induced by twisting of the linkages when the cannulas obtain a biopsy sample. That twisting results in a slight shortening of the overall length of the cannula comprising the linkages. This coupling of the inner and outer cannula drivers permit a relative movement of the cannulas commensurate with the dimensional change caused by twisting of the linkages.

This constraint in motion between the inner and outer cannulae created by the coupling of the cannulae at their distal ends is useful in limiting the extent to which the linkages 70 can shorten or expand when the biopsy device is being fired. The linkages are relatively fragile, and by limiting this range of motion, the linkages are less likely to break or otherwise become damaged. For example, when the biopsy device is fired, the dynamic impact may be strong enough to buckle the linkages when the cannulae stop moving, and/or to break the linkages during recoil after the cannulae reach their fully extended position.

In another embodiment, the cannula driver coupler 154 may be a spring located between the inner cannula driver and the outer cannula driver, where expansion of the spring provides a force that resists excessive movement of the inner cannula relative to the outer cannula. This restraint of movement precludes the distal ends of the inner and outer cannulae from moving too far with respect to one another in a longitudinal direction, such that the linkages 70 might break or rupture.

Once the device has been actuated and injected into tissue, and a biopsy sample captured by the needle set, the needle set may be withdrawn from the tissue site. To retrieve the biopsy sample from the needle set, the device may be cocked again to draw the needle set back. In embodiments including a rotating cannula driver, cocking the device after actuation causes the rotation of the cannula driver in a direction opposite to that which was caused by actuation. Post-actuation cocking also urges both cannula drivers distally. As inner and outer cannulae 242 and 244 are annularly arranged over the stylet portion of assembly 240 and as stylet assembly 240 remains stationary, the distal movement of cannula drivers 248 and 250 causes cannulae 242 and 244 to be drawn distally over the proximal end of stylet assembly 240, thereby exposing and releasing the biopsy sample.

In a preferred embodiment, the present invention provides a needle set for use in a biopsy device, where the needle set comprises two annularly nested cannulae, i.e., one cannula nests inside the lumen of the other cannula. Each of the two nested cannulae has a distal end, a proximal end, a lumen, and a cannula body. The two nested cannulae are joined at their proximal ends so that those proximal ends cannot independently move in either a rotational or longitudinal direction with respect to one another, for example, if one proximal end moves then the other proximal end must likewise move, and to the same degree or very nearly the same degree or else the two proximal ends will become unjoined. The distal ends of the at least two joined cannulae are mountable in a biopsy device and are preferably coupled together, i.e., the two distal ends can move independently from one another in both a longitudinal and rotational sense, however the coupling limits or constrains the extent of longitudinal and rotational movement that one distal end enjoys relative to the other distal end. The coupling may be achieved by having each of the cannula fixedly attached to a hub, i.e., the inner cannula is affixed to an inner cannula hub and the outer cannula is affixed to an outer cannula hub, so that each member of a hub-cannula pair moves together. The two hubs, each of which is fixed to its respective cannula, e.g., by gluing or bolting a hub to a cannula, can be coupled together, for example with a spring, thereby effectively coupling together the distal ends of the inner and outer cannulae. At least one of the two joined cannulae, and preferably the inner cannula, further have at least two flexible linkages joining the cannula body to its proximal end. Upon rotating the cannula body around its longitudinal axis while maintaining the proximal end in a fixed position, the linkages will contact one another, effectively cutting the luminal space within the cannula body at a location between the cannula body and the proximal end, thereby also cutting any material, for example biopsy material, present in the lumen.

Although the present invention has been shown and described in detail with regard to only a few exemplary embodiments of the invention, it should be understood by those skilled in the art that it is not intended to limit the invention to the specific embodiments disclosed. Various modifications, omissions, and additions may be made to the disclosed embodiments without materially departing from the novel teachings and advantages of the invention, particularly in light of the foregoing teachings. Accordingly, it is intended to cover all such modifications, omissions, additions, and equivalents as may be included within the spirit and scope of the invention as defined by the following claims.

What is claimed is:

1. A needle set for use in a biopsy device, wherein the needle set comprises a plurality of annularly nested cannulae,
a. each of said cannulae having a distal end, a proximal end, a lumen, and a cannula body, at least two of said cannulae being joined at their proximal ends,
b. the distal ends of the at least two joined cannulae being mountable in the biopsy device, and
c. at least one of the at least two joined cannulae further comprising at least two flexible linkages joining the cannula body to its proximal end.

2. The needle set of claim 1, wherein at least one cannula in the plurality of cannulae comprises a cutting edge at its proximal end.

3. The needle set of claim 1, further comprising a stylet annularly nested in the plurality of cannulae.

4. The needle set of claim 1, wherein the cannula comprising flexible linkages is disposed in the lumen of the cannula to which it is joined.

5. The needle set of claim 1, further comprising a therapeutic composition.

6. The needle set of claim 1 wherein the proximal ends of the at least two joined cannulae are coupled to one another.

7. The needle set of claim 6 wherein the proximal ends of the at least two joined cannulae are coupled to one another by way of a spring.

8. The cannula set of claim 6 wherein the at least two joined cannulae comprise an inner cannula and an outer cannula, where the inner cannula is at least partially located within the lumen of the outer cannula, where the inner cannula is fixedly attached to an inner cannula driver and the outer cannula is fixedly attached to an outer cannula driver, where the inner cannula driver and the outer cannula driver are joined together to provide constraint in movement of the distal end of the inner cannula relative to the distal end of the outer cannula.

9. The cannula set of claim 8 wherein the inner cannula driver and the outer cannula driver are coupled together and restrict movement of the distal end of the inner cannula relative to the distal end of the outer cannula.
